(19) United States
(12) Patent Application Publication
(10) Pub. No.: US 2025/0340522 A1
(43) Pub. Date: Nov. 6, 2025

(54) ALBICIDIN DERIVATIVES, THEIR USE AND SYNTHESIS

(71) Applicant: TECHNISCHE UNIVERSITÄT BERLIN, Berlin (DE)

(72) Inventors: Roderich SÜSSMUTH, Berlin (DE); John WESTON, Kelkheim (DE); Leonardo KLEEBAUER, Berlin (DE); Lieby ZBOROVSKY, Berlin (DE); Kay HOMMERNICK, Berlin (DE)

(73) Assignee: TECHNISCHE UNIVERSITÄT BERLIN, Berlin (DE)

(21) Appl. No.: 18/871,126

(22) PCT Filed: May 22, 2023
(86) PCT No.: PCT/EP2023/063580

§ 371 Date: Dec. 2, 2024

(30) Foreign Application Priority Data
May 30, 2022 (EP) 22176144.8

(51) Int. Cl. C07D 249/04; A61P 31/04

(52) U.S. Cl. CPC C07D 249/04; A61P 31/04

(57) ABSTRACT

It is provided a chemical compound according to general formula (1):

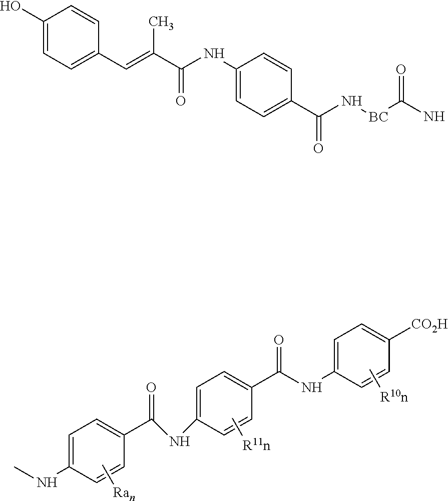

### BACKGROUND

Albicidin is a natural product, isolated from Xanthomonas albilineans and heterologously expressed in Xanthomonas axonopodis pv vesicatoria. Its structure (see below) is based on peptides and amino acids, but it does not contain any proteinogenic amino acids.

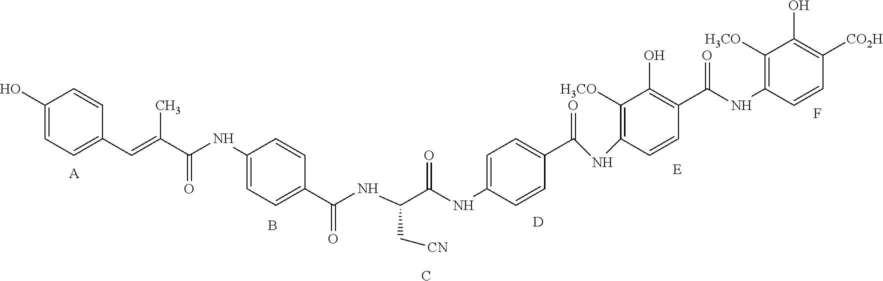

Albicidin is, on the one hand, a causative agent of the leaf scald disease in sugar cane and on the other hand a DNA-gyrase-inhibitor of prokaryotic cells (gram-positive and -negative). The mentioned properties make the natural product albicidin a potential antibiotic.

The known molecular structure of albicidin and available synthetic routes allows the development of a plurality of novel derivatives that may exhibit potential antimicrobial activities.

It is known that peptides as drugs can be hydrolysed relatively quickly by enzymes in the body, i.e. they are degraded. To counteract, some groups have been introduced that are supposed to reduce the rate of hydrolysis by increasing steric hindrance, to give compounds which are more stable in the presence of enzymes.

Many of the groups introduced but not all, which increase steric hindrance, are electron withdrawing in nature.

Some bacteria have already developed resistance to the natural product albicidin, and to counteract this, various chemical modifications have been made to the molecule. Specifically, the bacterium Pantoea dispersa is resistant to albicidin. An enzyme of the pathogen, more precisely the serine endopeptidase, is able to cleave albicidin between the building block D and E. The individual fragments have no antibacterial effect. Thus, it is desirable that the cleavage of the peptide bond should be prevented.

The bacterium Pantoea dispersa is a rare gram-negative microorganism that is mainly pathogenic to plants, but there are also known cases where humans have been infected with the pathogen. Treatment with the usual antibiotics: Clavulanic acid, piperacillin/tazobactam, all cephalosporins, all carbapenems, all aminoglycosides, fluoroquinolones and other antibiotics is ineffective. Only treatment with tigecycline (MIC=1) or colistin (MIC≤0.5) has been successful up to now. The consequences of no treatment or an ineffective treatment are progressive, systematic inflammatory reactions and even death.

Many of the groups introduced but not all, which increase steric hindrance and thus reduce the rate of hydrolysis, are rather lipophilic. Introducing lipophilic groups can be expected to increase the proportion of active substance bound to human serum, consequently the proportion of “free” active substance decreases and the concentration-dependent activity decreases. In this case it has been found that introducing such groups has little effect on the activity as measured. Accordingly, the antibacterial activity value (MIC) in the presence of human serum remains the same even when lipophilic substituents are introduced.

### SUMMARY

The problem underlying the proposed solution is therefore provision of new compounds, which comprise antibiotic properties, solubility and bioavailability, method of their synthesis and their use. This problem is attained by a compound having features as described herein.

### Terms and Definitions

The term “purity” as used in the context of the present specification with respect to a preparation of a certain compound refers to the content of said compound relative to the sum of all compounds contained in the preparation. The term “compound” in this context is to be understood as a compound according to the solution (or any specific embodiments thereof) as well as any salts, hydrates or solvates thereof. Thus, the respective salts, hydrates or solvates are not considered as impurities according to the previous definition. The “purity” of a compound may be determined using elemental analysis, HPLC analysis using UV diode array detection also in combination with mass spectrometry detection, or quantitative NMR analysis.

The term “substituted” refers to the addition of a substituent group to a parent moiety. “Substituent groups” can be protected or unprotected and can be added to one available site or to many available sites in a parent moiety.

### Description of the Solution

According to one aspect, the solution relates to compounds having a molecular structure as defined by formula (1)

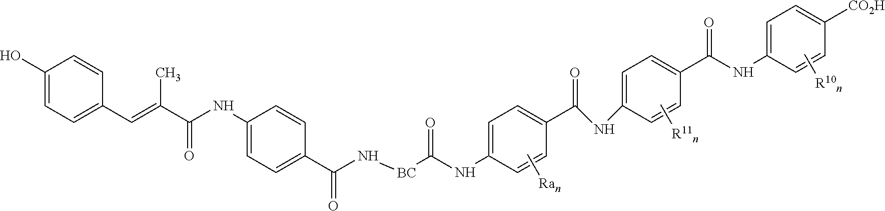

a) with BC being

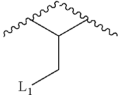

with L₁ being a substituted or unsubstituted C5-C6 aromatic heterocycle,
b) with n of R¹⁰ₙ and n of R¹¹ₙ being independently from each other 0, 1, 2, 3 or 4, in particular n of R¹⁰ₙ and n of R¹¹ₙ being 0, 1, 2 or 3, more particular n of R¹⁰ₙ and n of R¹¹ₙ being 1 and 2;
   with each R¹⁰ and R¹¹ being selected independently from any other R¹⁰ and R¹¹ from —OH, —F, —Cl, —Br, —I, —CCH, —CN, —OC₁-C₆ alkyl, in particular from —OH, —F, —OCH₃, —OC₂H₅, —OiC₃H₇, —OnC₃H₇, —OCF₃; and
c) with n of R^{a}ₙ being 1 or 2;
   with Rₐ being a substituent selected from halogen, in particular —Cl, —F, —Br, —I, —NO₂, —NH₂, —CO₂H, —CN, —CF₃.

In one embodiment, Rₐ being an electron withdrawing moiety is selected from —Cl, —F, —Br, —I, —CF₃, in particular from —Cl and —F.

In a further embodiment, R^{a} may be —F and n may be 1 or 2, preferably 2. In another embodiment, R^{a} may be —Cl and n may be 1.

In one embodiment, the moiety L₁ is a five membered aromatic N-heterocycle. In a preferred embodiment, moiety L₁ is an imidazole or triazole, most preferably an unsubstituted triazole.

Thus, in an embodiment the present compound may be of the general formulae (1a):

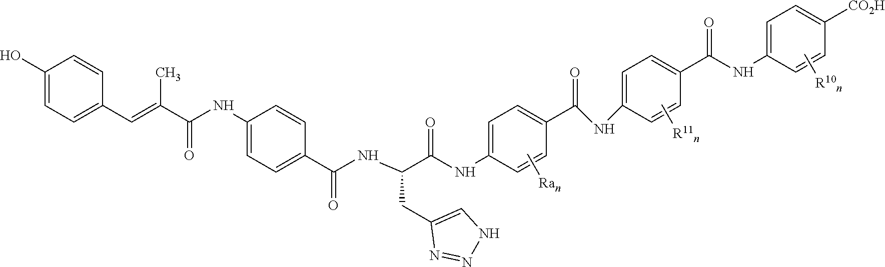

with BC, R^{a}ₙ, R¹⁰ₙ and R¹¹ₙ as described previously above.

In another preferred embodiment, n of R¹⁰ₙ and n of R¹¹ₙ being 0, 1, 2, 3 or 4, in particular n of R¹⁰ₙ and n of R¹¹, being 0, 1, 2 or 3, and with each R¹⁰ and with each R¹¹ independently from any other R¹⁰ being selected from —OH, —OCH₃, —OC₂H₅ or —OiPr, particularly with one R¹⁰ or R¹¹ being —OH and the other R¹⁰ or R¹¹ being —OCH₃, —OC₂H₅ or —OiPr respectively. In one specific embodiment R¹⁰ is H and R¹¹ is one of from —OH, —OCH₃, —OC₂H₅ or —OiPr.

In an embodiment the present compound may be of the general formulae (2)

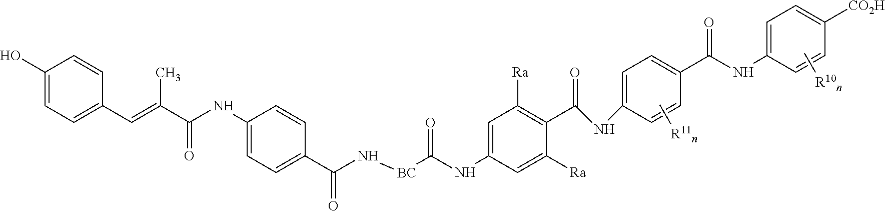

with R^{a} being a substituent selected from halogen, in particular —Cl, —F, —Br, —I, —NO₂, —NH₂, —CO₂H, —CN, —CF₃, preferably —Cl, —F, —Br, —I, —CF₃, in particular preferably from —Cl and —F,
with BC, R¹⁰ₙ and R¹¹ₙ as described previously above.

In another embodiment the present compound may be of the general formulae (3)

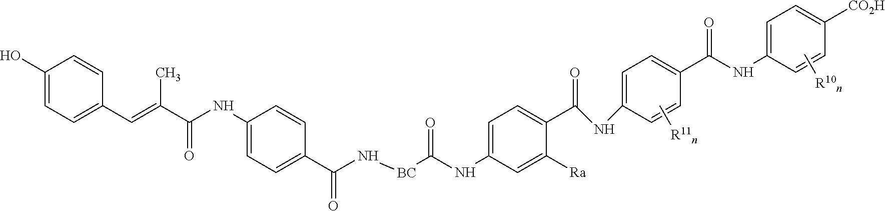

with R^{a} being a substituent selected from halogen, in particular —Cl, —F, —Br, —I, —NO₂, —NH₂, —CO₂H, —CN, —CF₃, preferably —Cl, —F, —Br, —I, —CF₃, in particular preferably from —Cl and —F,
with BC, R¹⁰ₙ and R¹¹ₙ as described previously above.

In a further embodiment the present compound may be of the general formulae (4)

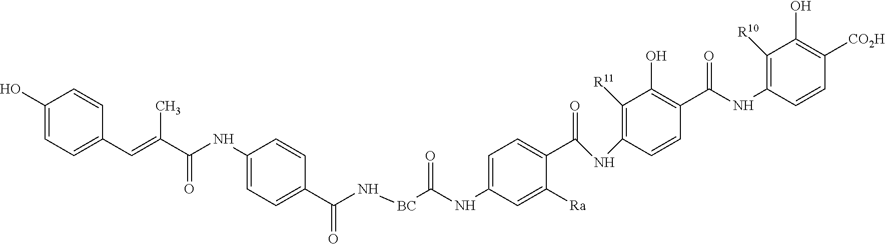

with R^{a} being a substituent selected from halogen, in particular —Cl, —F, —Br, —I, —NO₂, —NH₂, —CO₂H, —CN, —CF₃, preferably —Cl, —F, —Br, —I, —CF₃, in particular preferably from —Cl and —F,
with BC, R¹⁰ and R¹¹ as described previously above.

In still another embodiment the present compound may be of the general formulae (5)

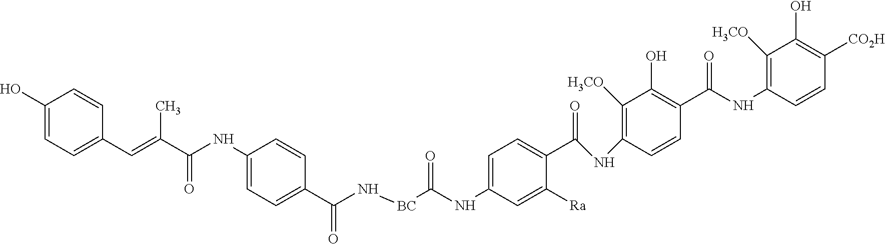

with R^{a} being a substituent selected from halogen, in particular —Cl, —F, —Br, —I, —NO₂, —NH₂, —CO₂H, —CN, —CF₃, preferably —Cl, —F, —Br, —I, —CF₃, in particular preferably from —Cl and —F,
with BC as described previously above.

In still another embodiment the present compound may be of the general formulae (6)

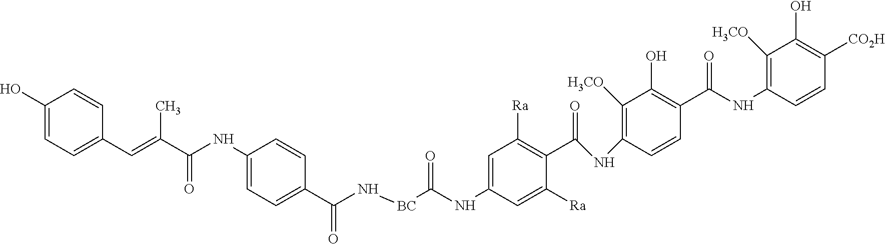

with R^{a} being an electron withdrawing moiety selected from —Cl, —F, —Br, —I, —CF₃, in particular preferably from —Cl and —F,
with BC as described previously above.

Particular embodiments of the solution are one of the following compounds:

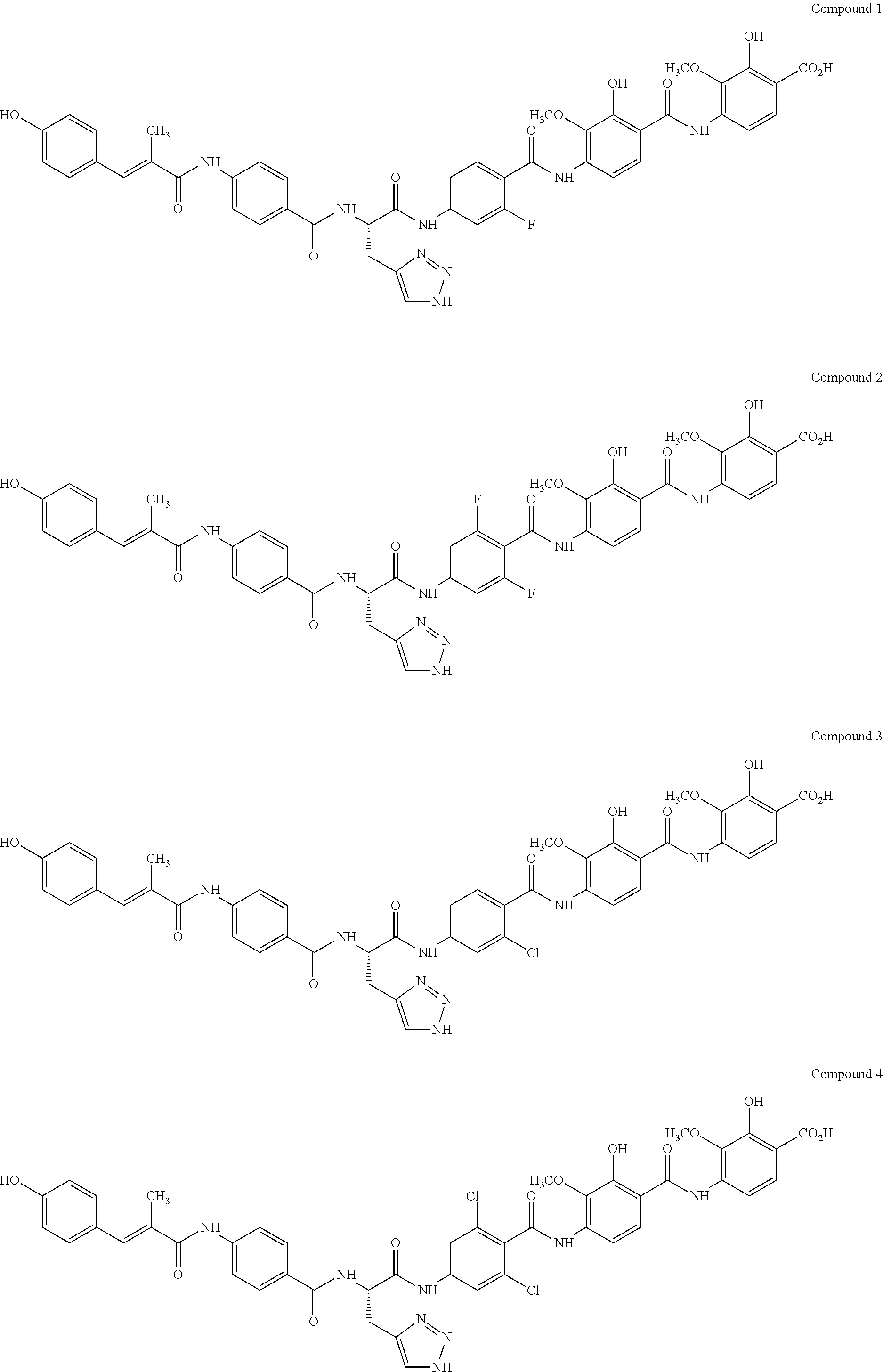

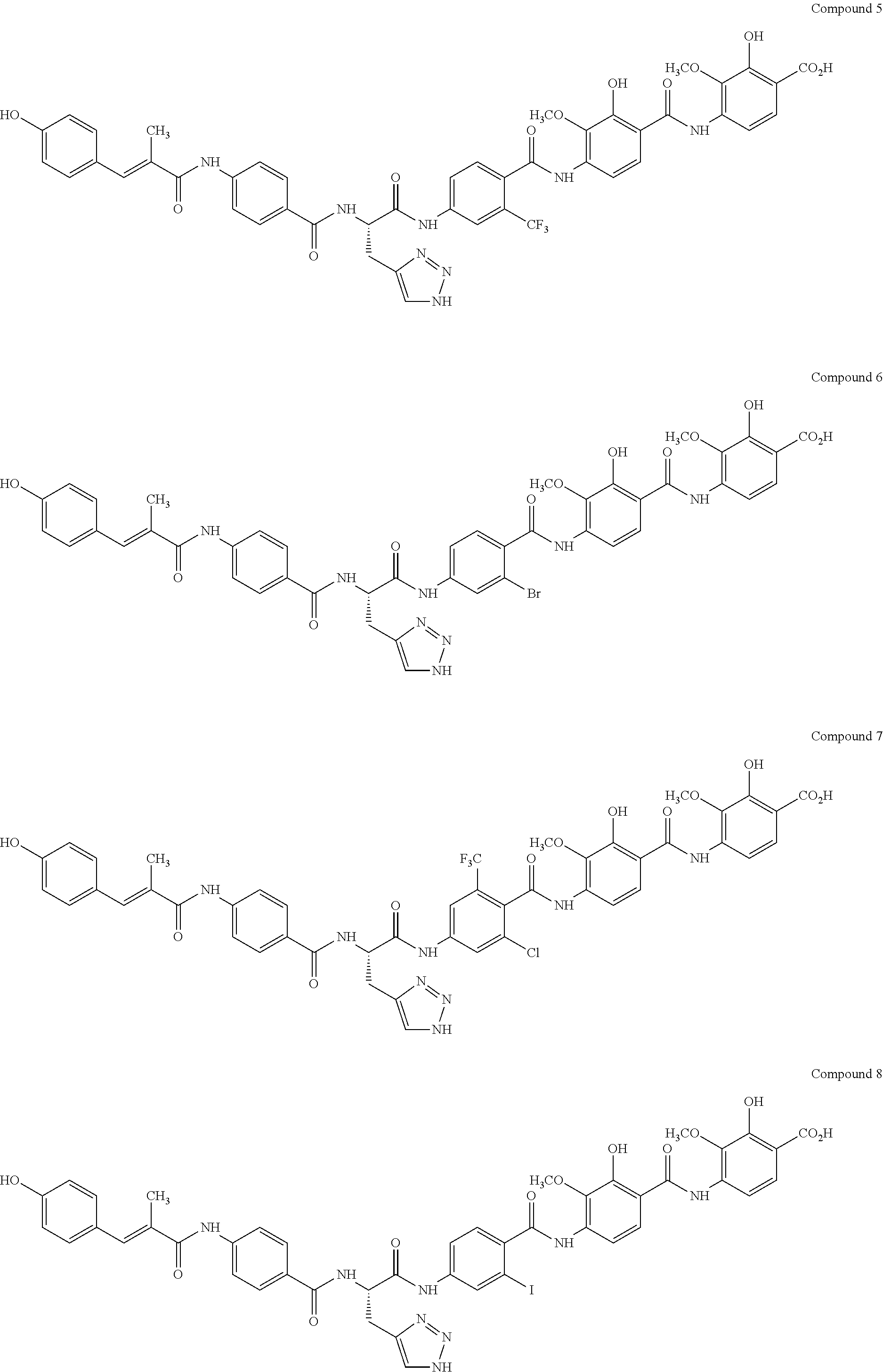

The compounds of the present solution may be used in a method of treatment of diseases, in particular for use in a method of treatment of bacterial infections caused by gram-negative or gram-positive bacterial strains.

The bacterial infection may be an infection (by a gram-negative bacterium) caused by one of the genus Acinetobacter, Bordatella, Borellia, Brucella, Camphylobacter, Chlamydia, Chlamydophila, Enterobacter, Escherichia, Francisella, Haemophilus, Helicobacter, Klebsiella, Legionella, Leptospira, Morganella Moraxella, Neisseria, Proteus, Pseudomonas, Rickettsia, Shigella, Salmonella, Stenotrophomonas, Treponema or Yersinia, in particular an infection caused by one of the genus Escherichia, Enterobacter, Salmonella, Klebsiella, Pseudomonas, Haemophilus, Shigella, Proteus or Morganella.

In a further embodiment, the bacterial infection is an infection caused
by a gram-positive bacterium, particularly an infection by one of the genus Bacillus, Clostridium, Corynebacterium, Enterococcus, Listeria, Micrococcus, Staphylococcus or Streptococcus, further in particular by one of the genus of Staphylococcus, Streptococcus, Bacillus or Micrococcus or
by a bacterium of the family of Mycobacteriaceae, in particular of the genus Mycobacterium, further in particular an infection by one of Mycobacterium tuberculosis, Mycobacterium leprae, Mycobacterium ulcerans or Mycobacterium avium, or
by a bacterium of the family of Mycoplasmataceae, in particular of the genus Mycoplasma, further in particular an infection by Mycoplasma pneumonia.

For this purpose, the present compounds may be provided in a pharmaceutical acceptable form. Pharmaceutically acceptable salts of the present compounds mean both their organic and inorganic salts as described in Remington's Pharmaceutical Sciences (17th edition, page 1418 (1985)). Because of the physical and chemical stability and the solubility, preference is given for acidic groups inter alia to sodium, potassium, calcium and ammonium salts; preference is given for basic groups inter alia to salts of maleic acid, fumaric acid, succinic acid, malic acid, tartaric acid, methylsulfonic acid, hydrochloric acid, sulfuric acid, phosphoric acid or of carboxylic acids or sulfonic acids, for example as hydrochlorides, hydrobromides, phosphates, sulfates, methanesulfonates, acetates, lactates, maleates, fumarates, malates, gluconates, and salts of amino acids, of natural bases or carboxylic acids. The preparation of pharmaceutically acceptable salts from compounds of the formula (I) which are capable of salt formation, including their stereoisomeric forms, takes place in a manner known per se. The present compounds form stable alkali metal, alkaline earth metal or optionally substituted ammonium salts with basic reagents such as hydroxides, carbonates, bicarbonates, alcoholates and ammonia or organic bases, for example trimethyl- or triethylamine, ethanolamine, diethanolamine or triethanolamine, trometamol or else basic amino acids, for example lysine, ornithine or arginine. Where the compounds of the formula (I) have basic groups, stable acid addition salts can also be prepared with strong acids. Suitable pharmaceutically acceptable acid addition salts of the compounds of the solution are salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. The hydrochloride salt is a preferred salt.

In a preferred embodiment formulations of the present albicidin derivatives are provided which contain cyclodextrins for improving solubility of the otherwise poorly soluble albicidin derivatives. Cyclodextrins are used in a concentration of 20-40%, preferably 25-35%, more preferably 28-30%.

Salts with a pharmaceutically unacceptable anion such as, for example, trifluoroacetate likewise belong within the framework of the solution as useful intermediates for the preparation or purification of pharmaceutically acceptable salts and/or for use in non-therapeutic, for example in vitro, applications.

The solution furthermore relates to pharmaceutical preparations (or pharmaceutical compositions) which contain an effective amount of at least one of the present compounds and/or its pharmaceutically acceptable salts and a pharmaceutically acceptable carrier, i.e. one or more pharmaceutically acceptable carrier substances (or vehicles) and/or additives (or excipients). The pharmaceuticals can be administered orally, for example in the form of pills, tablets, lacquered tablets, coated tablets, granules, hard and soft gelatine capsules, solutions, syrups, emulsions, suspensions or aerosol mixtures. Administration, however, can also be carried out rectally, for example in the form of suppositories, or parenterally, for example intravenously, intramuscularly or subcutaneously, in the form of injection solutions or infusion solutions, microcapsules, implants or rods, or percutaneously or topically, for example in the form of ointments, solutions or tinctures, or in other ways, for example in the form of aerosols or nasal sprays.

The pharmaceutical preparations according to the solution are prepared in a manner known per se and familiar to one skilled in the art, pharmaceutically acceptable inert inorganic and/or organic carrier substances and/or additives being used in addition to the compound(s) of the formula (I) and/or its (their) pharmaceutically acceptable salts and/or its (their) prodrugs. For the production of pills, tablets, coated tablets and hard gelatine capsules it is possible to use, for example, lactose, corn starch or derivatives thereof, talc, stearic acid or its salts, etc. Carrier substances for soft gelatine capsules and suppositories are, for example, fats, waxes, semisolid and liquid polyols, natural or hardened oils, etc. Suitable carrier substances for the production of solutions, for example injection solutions, or of emulsions or syrups are, for example, water, saline, alcohols, glycerol, polyols, sucrose, invert sugar, glucose, vegetable oils, etc. Suitable carrier substances for microcapsules, implants or rods are, for example, copolymers of glycolic acid and lactic acid. The pharmaceutical preparations normally contain about 0.5 to about 90% by weight of the present compounds and/or their pharmaceutically acceptable salts and/or their prodrugs. The amount of the active ingredient of the formula (I) and/or its pharmaceutically acceptable salts and/or its prodrugs in the pharmaceutical preparations normally is from about 0.5 to about 1000 mg, preferably from about 1 to about 500 mg.

A prodrug is a precursor chemical compound of a biological active compound of the solution. Instead of administering the active compound or drug, a prodrug might be used instead to improve the absorption, distribution, metabolization and excretion. Prodrugs are often designed to improve bioavailability when a drug itself is poorly absorbed from the gastrointestinal tract. A prodrug may also be used to improve the selectively of the drug. This reduces adverse or unintended effects of a drug, especially important in treatments like chemotherapy, which can have severe unintended and undesirable side effects.

In addition to the active compound according to the solution and/or their pharmaceutically acceptable salts and to carrier substances, the pharmaceutical preparations can contain one or more additives such as, for example, fillers, disintegrants, binders, lubricants, wetting agents, stabilizers, emulsifiers, preservatives, sweeteners, colorants, flavourings, aromatizers, thickeners, diluents, buffer substances, solvents, solubilizers, agents for achieving a depot effect, salts for altering the osmotic pressure, coating agents or antioxidants. They can also contain two or more of the present compounds and/or their pharmaceutically acceptable salts. In case a pharmaceutical preparation contains two or more of the present compounds the selection of the individual compounds can aim at a specific overall pharmacological profile of the pharmaceutical preparation. For example, a highly potent compound with a shorter duration of action may be combined with a long-acting compound of lower potency. The flexibility permitted with respect to the choice of substituents in the present compounds allows a great deal of control over the biological and physico-chemical properties of the compounds and thus allows the selection of such desired compounds. Furthermore, in addition to at least one compound and/or its pharmaceutically acceptable salts, the pharmaceutical preparations can also contain one or more other therapeutically or prophylactically active ingredients. When using the present compounds the dose can vary within wide limits and, as is customary and is known to the physician, is to be suited to the individual conditions in each individual case. It depends, for example, on the specific compound employed, on the nature and severity of the disease to be treated, on the mode and the schedule of administration, or on whether an acute or chronic condition is treated or whether prophylaxis is carried out. An appropriate dosage can be established using clinical approaches well known in the medical art. In general, the daily dose for achieving the desired results in an adult weighing about 75 kg is from about 0.01 to about 100 mg/kg, preferably from about 0.1 to about 50 mg/kg, in particular from about 0.1 to about 10 mg/kg, (in each case in mg per kg of body weight). The daily dose can be divided, in particular in the case of the administration of relatively large amounts, into several, for example 2, 3 or 4, part administrations. As usual, depending on individual behaviour it may be necessary to deviate upwards or downwards from the daily dose indicated.

The compounds of the solution may also exist in various polymorphous forms, for example as amorphous and crystalline polymorphous forms. All polymorphous forms of the compounds of the solution belong within the framework of the solution and are a further aspect of the solution.

The compounds of the present solution may be present as optical isomers or as mixtures thereof. The solution relates both to the pure isomers and all possible isomeric mixtures and is hereinafter understood as doing so, even if stereochemical details are not specifically mentioned in every case. Enantiomeric mixtures of compounds of the general formula 1, which are obtainable by the process or any other way, may be separated in known manner-on the basis of the physical-chemical differences of their components-into pure enantiomers, for example by fractional crystallisation, distillation and/or chromatography, in particular by preparative HPLC using a chiral HPLC column.

According to the solution, apart from separation of corresponding isomer mixtures, generally known methods of diastereoselective or enantioselective synthesis can also be applied to obtain pure diastereoisomers or enantiomers, e.g. by carrying out the method described hereinafter and using educts with correspondingly suitable stereochemistry.

It is advantageous to isolate or synthesise the biologically more active isomer, provided that the individual compounds have different biological activities.

The solution is explained in more detail by means of the following examples.

### Methods of synthesis

One general procedure for the synthesis of albicidin-derivatives with variations to amide bonds may comprise the steps according to the following procedure.

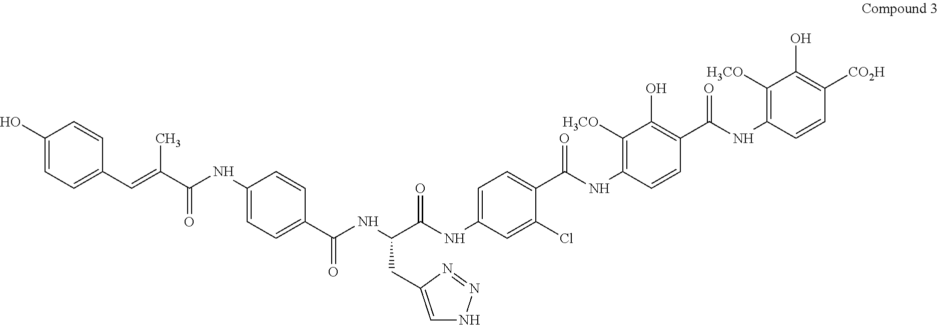 [SMILES: COc1c(NC(=O)c2ccc(NC(=O)c3ccc(NC(=O)[C@H](Cc4c[nH]nn4)NC(=O)c4ccc(NC(=O)/C(C)=C/c5ccc(O)cc5)cc4)cc3Cl)c(OC)c2O)ccc(C(=O)O)c1O]

Compound 3 is synthesized in a multistep synthesis route as follows:

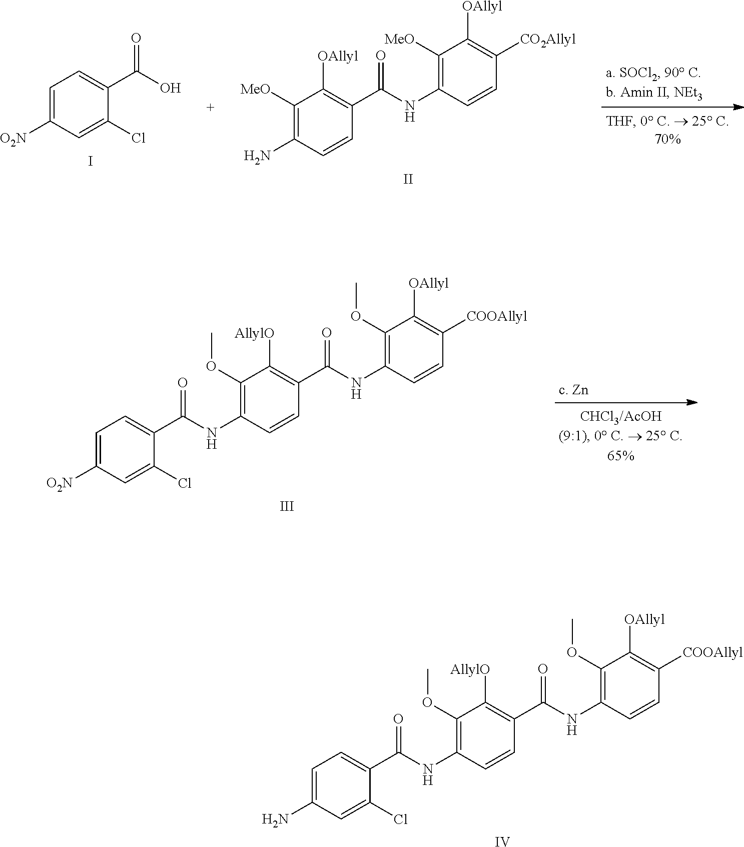

### Preparation of Compound III

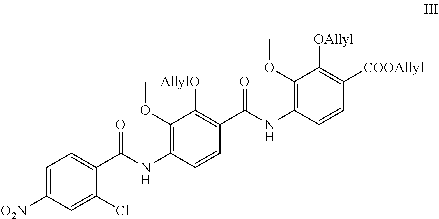

The commercially available 2-Chloro-4-nitrobenzoic acid I (348 mg, 743 μmol, 1.40 eq.) was dissolved in SOCl₂ (5 mL) and refluxed for 2 h. All volatiles were removed and the residue dissolved in THF (5 mL). The prepared acid chloride solution was added at −15° C. to a prepared solution of the literature known amine II (348 mg, 743 μmol, 1.00 eq.) in anhydrous THF (24 mL) and triethylamine (207 μL, 1.49 mmol, 2.00 eq.). The reaction mixture was stirred for 20 minutes and diluted with diethyl ether (22 ml). The solid was filtered, washed with diethyl ether (3×50 ml) and dried in vacuo to yield III (338 mg, 70%) as a yellow solid.

¹H NMR (500 MHZ, DMSO-d₆) δ 10.62 (s, 1H), 10.53 (s, 1H), 8.41 (d, J=2.2 Hz, 1H), 8.35-8.27 (m, 2H), 8.11 (d, J=8.8 Hz, 1H), 7.90 (d, J=8.4 Hz, 1H), 7.83 (d, J=8.8 Hz, 1H), 7.57 (d, J=8.8 Hz, 1H), 6.06 (dddt, J=27.9, 16.1, 10.7, 5.3 Hz, 3H), 5.40 (ddd, J=18.2, 16.1, 2.1 Hz, 3H), 5.31-5.21 (m, 3H), 4.78 (dd, J=11.3, 5.9 Hz, 4H), 4.54 (d, J=5.8 Hz, 2H), 3.93 (s, 3H), 3.87 (s, 3H).

¹³C NMR (126 MHz, DMSO-d₆) δ 164.4, 151.0, 149.7, 148.3, 142.5, 136.4, 135.8, 133.9, 132.7, 132.6, 131.1, 130.2, 126.2, 125.7, 124.4, 122.3, 120.3, 120.3, 120.1, 118.1, 117.8, 114.8, 75.1, 74.5, 65.1, 61.1, 61.0.

HRMS (ESI-ORBITRAP): m/z [M+H]⁺ ber. für C₃₂H₃₁ClN₃O₁₀: 652.1692; gef. 652.1696.

### Preparation of Compound IV

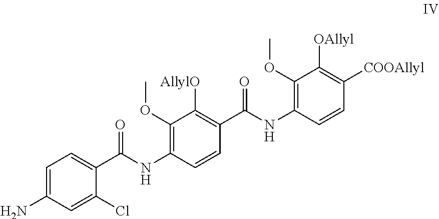

Compound III (331 mg, 508 μmol, 1.00 eq.) was suspended in a mixture of chloroform (90 ml) and acetic acid (10 ml) and cooled to 0° C. Zinc dust (664 mg, 10.2 mmol, 20.0 eq.) was added portion wise. After 20 min the reaction was proven to be complete (verified by TLC-control). The solid was filtered and washed with DCM (3×100 ml). The combined liquids were evaporated to dryness. The residue was taken up in DCM (300 ml) and saturated aqueous NaHCO₃—Solution (300 ml). The aqueous phase was further extracted twice with DCM (2×100 ml). The combined organic fractions were washed successively with saturated aqueous NaHCO₃—Solution (1×30 ml), distilled water (1×30 ml) and brine (1×30 ml), dried over Na₂SO₄ and evaporated to obtain IV (206 mg, 65%) as a yellow solid.

¹H NMR (400 MHZ, DMSO-d₆) δ 10.63 (s, 1H), 9.61 (s, 1H), 8.36-8.29 (m, 1H), 8.17 (d, J=8.9 Hz, 1H), 7.81 (d, J=8.9 Hz, 1H), 7.57 (d, J=8.8 Hz, 1H), 7.51 (d, J=8.5 Hz, 1H), 6.67 (d, J=2.2 Hz, 1H), 6.59 (dd, J=8.5, 2.2 Hz, 1H), 6.17-6.08 (m, 2H), 6.08-5.96 (m, 3H), 5.40 (ddq, J=17.2, 12.1, 1.7 Hz, 3H), 5.32-5.20 (m, 3H), 4.78 (ddt, J=7.0, 5.5, 1.3 Hz, 4H), 4.54 (dt, J=5.7, 1.4 Hz, 2H), 3.92 (s, 3H), 3.91 (s, 3H).

(¹H,¹³C)-HSQC_ed NMR (101 MHZ, DMSO-d₆) δ 115.3, 116.9, 126.5, 126.6, 126.8, 132.9, 114.2, 112.6, 133.8, 119.4, 119.8, 119.5, 118.6, 75.5, 65.5, 75.0, 61.4.

HRMS (ESI-ORBITRAP): m/z [M+H]⁺ ber. für C32H₃₃ClN₃O₈: 622.1951; gef. 622.1952.

### Preparation of Compound VI

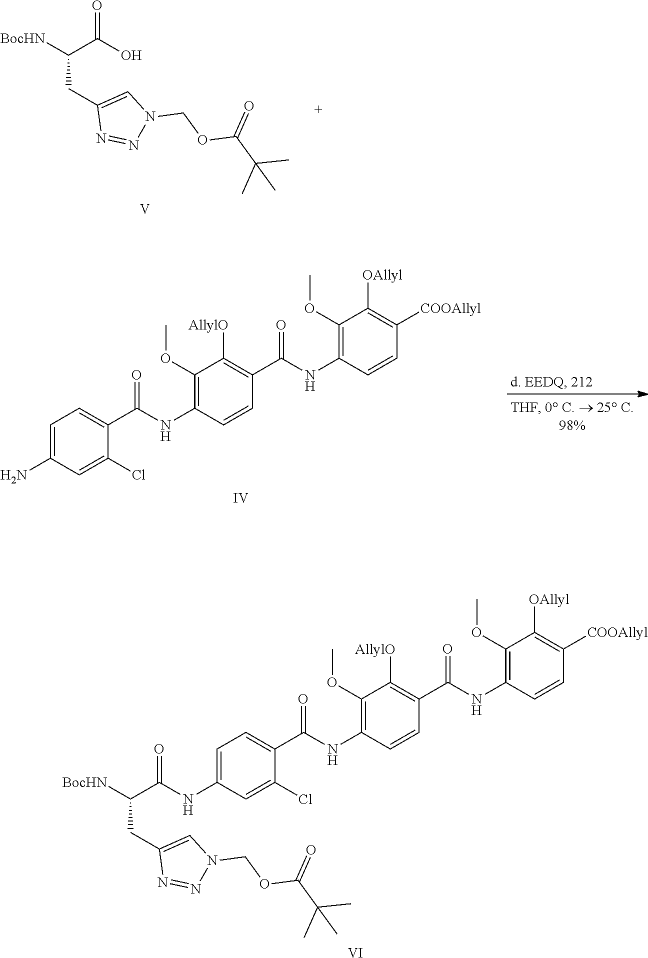

Literature known Boc-β-(1-pivaloyloxymethyl)-1,2,3-triazol-4-yl)-Alanine V (237 mg, 640 μmol, 2.00 eq.) was dissolved in THF (20 ml) and cooled to 0° C. N-Ethoxycarbonyl-2-ethoxy-1,2-dihydroqinoline (EEDQ) (237 mg, 960 μmol, 3.00 eq.) was added and after 5 minutes compound IV (199 mg, 320 μmol, 1.00 eq.) was added. The reaction mixture was slowly warmed to room temperature and stirred for 16 h. All volatiles were removed in vacuo and the residue was taken up in ethyl acetate (100 ml). The organic fraction was washed with saturated aqueous NaHCO₃—Solution (3×50 ml) and brine (1×50 ml), dried over Na₂SO₄ and evaporated. The residue was purified via flash chromatography on silica gel eluting with 1-15% acetone in DCM. Compound VI (305 mg, 98%) was obtained as a colourless solid.

¹H NMR (500 MHZ, DMSO-d₆) δ 10.63 (s, 1H), 10.45 (s, 1H), 9.99 (s, 1H), 8.33 (d, J=8.8 Hz, 1H), 8.08 (d, J=9.2 Hz, 1H), 7.98 (s, 1H), 7.91 (d, J=1.9 Hz, 1H), 7.81 (d, J=8.9 Hz, 1H), 7.61 (d, J=8.4 Hz, 1H), 7.57 (d, J=8.7 Hz, 2H), 7.22 (d, J=7.9 Hz, 1H), 6.29 (s, 2H), 6.07 (dddd, J=33.5, 22.5, 10.4, 4.6 Hz, 3H), 5.45-5.36 (m, 3H), 5.30-5.21 (m, 3H), 4.78 (t, J=7.4 Hz, 4H), 4.54 (d, J=5.7 Hz, 2H), 4.37 (d, J=6.2 Hz, 1H), 3.93 (s, 3H), 3.89 (s, 3H), 3.15-3.08 (m, 1H), 3.00 (dd, J=14.7, 9.2 Hz, 1H), 1.36 (s, 9H), 1.10 (s, 9H).

¹³C NMR (126 MHZ, DMSO-d₆) δ 115.6, 119.0, 124.9, 120.3, 126.7, 131.2, 119.0, 126.7, 69.9, 134.9, 119.8, 119.0, 75.9, 65.8, 75.2, 61.6, 61.7, 28.5, 26.9.

HRMS (ESI-ORBITRAP): m/z [M+H]⁺ ber. für C₄₈H₅₇ClN₇O₁₃: 974.3697; gef. 974.3688.

### Preparation of Compound VII

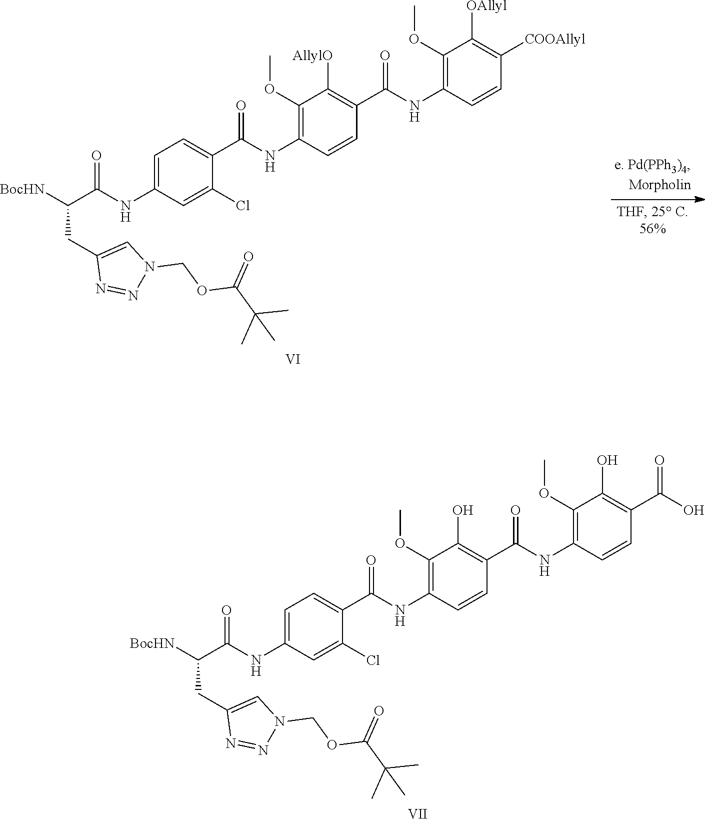

Tetrapeptide VI (220 mg, 226 μmol, 1.00 eq.) was dissolved in THF (5 ml) and morpholine (390 μL mL, 4.52 mmol, 20.0 eq.) and tetrakis(triphenylphosphin)palladium(0) (104 mg, 90.3 μmol, 0.40 eq.) were added. The mixture was stirred for 2.5 h shielded from light. All volatiles were removed in vacuo and the residue was purified via flash chromatography on C-18-material eluting with 5 to 50% acetonitrile in water. Compound VII (108 mg, 56%) was obtained as a white solid.

¹H NMR (500 MHZ, DMSO-d₆) δ 11.57 (s, 1H), 10.91 (s, 1H), 10.45 (s, 1H), 9.93 (s, 1H), 7.98 (s, 1H), 7.90 (d, J=1.9 Hz, 1H), 7.81 (d, J=8.9 Hz, 1H), 7.74 (s, 2H), 7.60 (d, J=8.4 Hz, 1H), 7.56 (dd, J=8.4, 1.9 Hz, 1H), 7.48 (d, J=8.6 Hz, 1H), 7.22 (d, J=7.9 Hz, 1H), 6.29 (s, 2H), 4.42-4.32 (m, 1H), 3.87 (s, 3H), 3.77 (s, 3H), 3.12 (dd, J=14.7, 5.0 Hz, 1H), 3.00 (dd, J=14.8, 9.3 Hz, 1H), 1.36 (s, 9H), 1.10 (s, 9H).

(¹H,¹³C)-HSQC_ed NMR (126 MHZ, DMSO-d₆) δ 124.6, 120.0, 125.4, 130.7, 118.0, 125.4, 70.2, 60.2, 61.2, 28.7, 26.9.

HRMS (ESI-ORBITRAP): m/z [M+H]⁺ ber. für C₃₉H₄₅ClN₇O₁₃: 854.2758; gef. 854.2758.

### Preparation of Compound VIII

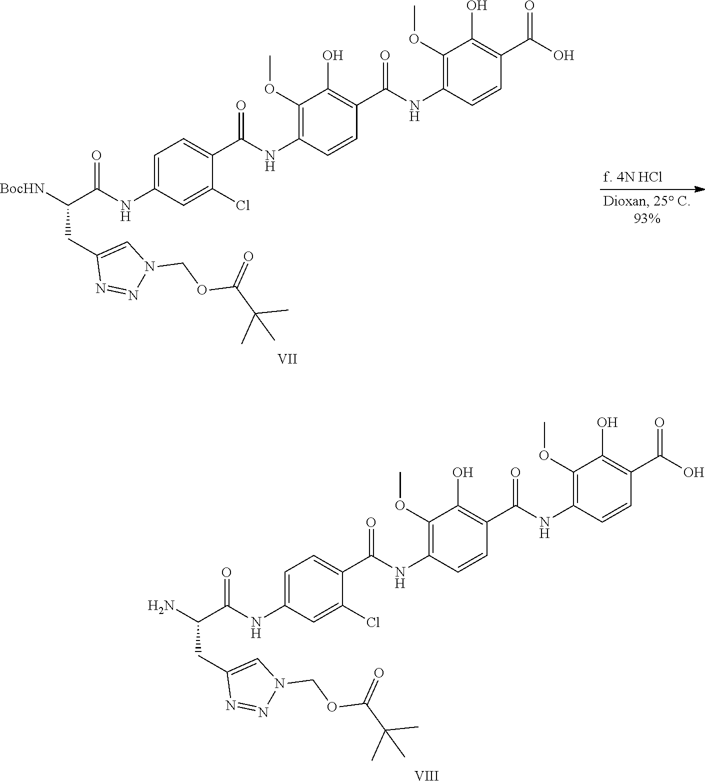

Tetrapeptide VII (108 mg, 126 μmol, 1.00 eq.) was dissolved 4 N HCl in dioxane and stirred for 1 hour. The solvent was evaporated in vacuo and the product VIII (94 mg, 93%) was obtained as white solid.

¹H NMR (500 MHZ, DMSO-d₆) δ 11.53 (s, 1H), 11.16 (s, 1H), 10.02 (s, 1H), 8.48 (s, 2H), 8.14 (s, 1H), 8.04 (d, J=8.9 Hz, 1H), 7.87-7.80 (m, 2H), 7.76 (s, 1H), 7.63 (d, J=8.4 Hz, 1H), 7.57 (dd, J=16.8, 9.0 Hz, 2H), 6.29 (s, 2H), 6.26 (d, J=4.7 Hz, 1H), 4.33 (d, J=13.4 Hz, 1H), 3.91 (s, 3H), 3.77 (s, 3H), 3.69 (dd, J=16.5, 5.2 Hz, 1H), 3.50-3.46 (m, 1H), 1.10 (s, 9H).

(¹H,¹³C)-HSQC_ed NMR (126 MHZ, DMSO-d₆) δ 125.5, 124.4, 109.3, 119.1, 125.5, 95.7, 71.1, 61.3, 61.8, 26.6.

HRMS (ESI-ORBITRAP): m/z [M+H]⁺ ber. für C₃₄H₃₇ClN₇O₁₁: 754.2234; gef. 754.2230.

### Preparation of Compound 3

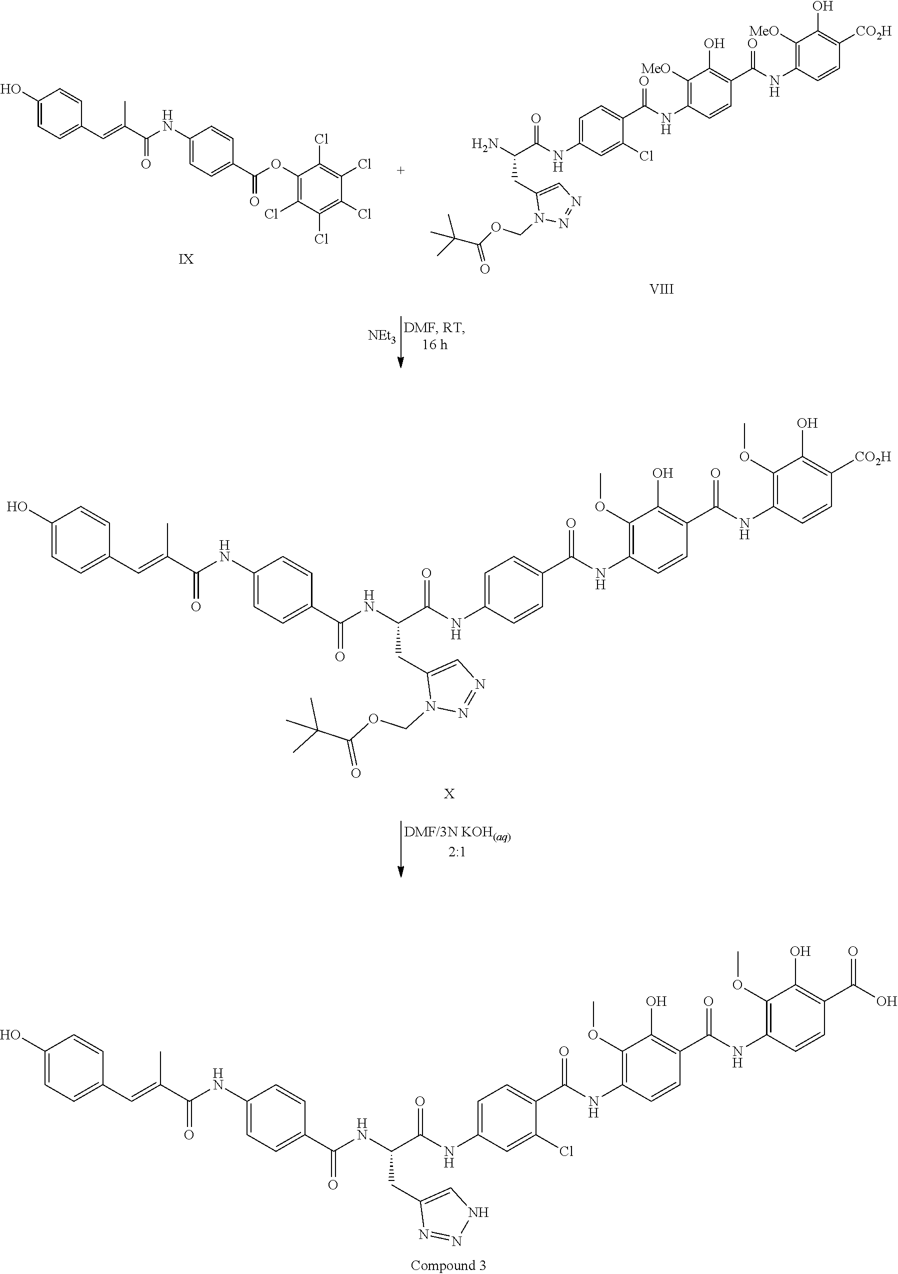

The tetrapeptide VIII (45.0 mg, 60 μmol, 1.00 eq.) was dissolved in DMF (2 ml) and triethylamine (83 μL, 10.0 eq.) was added. After adding the literature known active ester IX (39 mg, 65.6 μmol, 1.10 eq.), the mixture was stirred for 16 h. Then cooled to 0° C. and 3 N KOH_{(aq)} (1 ml) was added dropwise. After 15 min of stirring, 550 μl of 6 N HCl_{(aq)} were added dropwise. The resulting mixture was evaporated to dryness. The residue was purified via prep HPLC. Compound 3 (10.4 mg, 18%) was obtained as a white fluffy solid.

RP-HPLC: 38-52% MeCN (+0.1% TFA) in milliQ H₂O (+0.1% TFA); 30 min; 60 mL/min. [α]_{D}²⁴=+32° (c 0.08, DMSO).

¹H NMR (700 MHZ, DMSO-d₆) δ 11.57 (s, 1H), 11.52 (d, J=18.0 Hz, 1H), 11.18 (s, 1H), 10.53 (s, 1H), 10.08 (s, 1H), 10.02 (d, J=5.9 Hz, 1H), 9.76 (s, 1H), 8.71 (d, J=7.6 Hz, 1H), 8.06 (dd, J=8.9, 5.2 Hz, 1H), 7.92 (d, J=1.7 Hz, 1H), 7.87 (d, J=8.6 Hz, 2H), 7.84-7.79 (m, 3H), 7.76 (s, 1H), 7.67 (s, 1H), 7.63-7.57 (m, 3H), 7.35 (d, J=8.5 Hz, 2H), 7.26 (s, 1H), 6.84 (d, J=8.3 Hz, 2H), 4.87 (q, J=7.3 Hz, 1H), 3.91 (s, 3H), 3.76 (s, 3H), 3.29 (dd, J=14.7, 5.8 Hz, 1H), 3.23 (dd, J=14.8, 9.0 Hz, 1H), 2.11 (d, J=1.3 Hz, 3H).

(¹H,¹³C)-HSQC_ed NMR (176 MHZ, DMSO-d₆) δ 110.7, 120.0, 128.7, 128.7, 119.5, 126.2, 114.1, 130.5, 118.0, 126.2, 131.8, 134.4, 115.8, 54.8, 60.7, 61.3, 15.0.

HRMS (ESI-ORBITRAP): m/z [M+H]⁺ ber. für C₄₅H₄₀ClN₈O₁₂: 919.2449, gef. 919.2450, R_{T}=8.26 min.

The following compounds are obtained in analogous synthesis procedures.

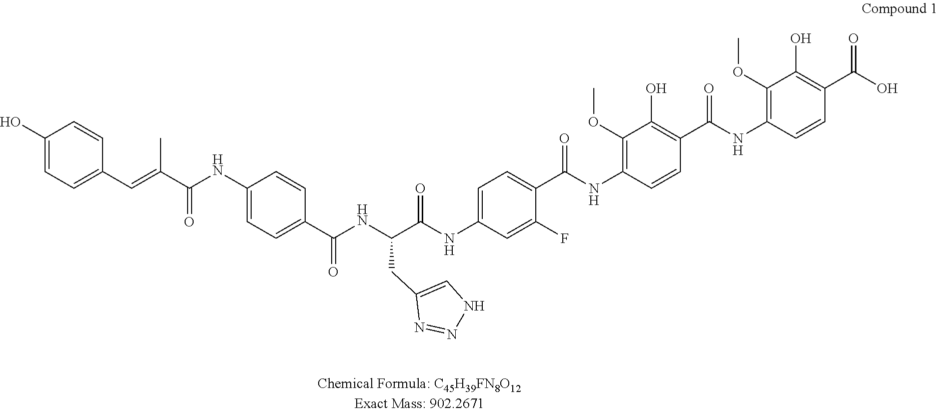 [SMILES: COc1c(NC(=O)c2ccc(NC(=O)c3ccc(NC(=O)[C@H](Cc4c[nH]nn4)NC(=O)c4ccc(NC(=O)/C(C)=C/c5ccc(O)cc5)cc4)cc3F)c(OC)c2O)ccc(C(=O)O)c1O]

RP-HPLC: 35-58% MeCN (+0.1% TFA) in milliQ H₂O (+0.1% TFA); 35 min; 70 mL/min. [α]_{D}²⁴=+27.0° (c 0.11, DMSO).

¹H NMR (500 MHZ, DMSO-d₆) δ 11.64 (s, 1H), 11.11 (s, 1H), 10.71 (s, 1H), 10.07 (s, 1H), 9.76 (s, 1H), 9.61 (d, J=9.6 Hz, 1H), 8.72 (s, 1H), 8.09-7.76 (m, 10H), 7.68 (s, 1H), 7.57 (d, J=8.7 Hz, 1H), 7.53-7.47 (m, 1H), 7.35 (d, J=8.8 Hz, 2H), 7.26 (s, 1H), 6.87-6.81 (m, 2H), 4.89 (dd, J=14.9, 5.0 Hz, 1H), 3.91 (s, 3H), 3.82 (s, 3H), 3.23 (d, J=13.8 Hz, 2H), 2.11 (s, 3H).

¹⁹F NMR (471 MHZ, DMSO-d₆) δ −111.10 (d, J=8.9 Hz).

(¹H,¹³C)-HSQC_ed NMR (126 MHZ, DMSO-d₆) δ 110.6, 112.7, 132.3, 128.6, 119.5, 106.6, 106.4, 126.0, 115.7, 131.8, 134.4, 115.9, 54.8, 60.6, 61.1, 40.3, 15.0.

HRMS (ESI-ORBITRAP): m/z [M+H]⁺ ber. für C₄₅H₄₀FN₈O₁₂: 903.2744, gef. 903.2746, R_{T}=8.19 min.

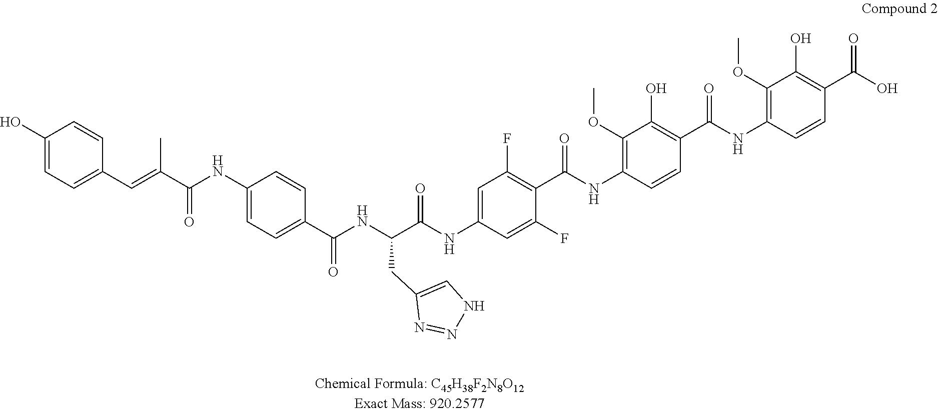 [SMILES: COc1c(NC(=O)c2ccc(NC(=O)c3c(F)cc(NC(=O)[C@H](Cc4c[nH]nn4)NC(=O)c4ccc(NC(=O)/C(C)=C/c5ccc(O)cc5)cc4)cc3F)c(OC)c2O)ccc(C(=O)O)c1O]

RP-HPLC: 40-53% MeCN (+0.1% TFA) in milliQ H₂O (+0.1% TFA); 30 min; 60 mL/min. [α]_{D}²⁴=+85° (c 0.07, DMSO).

¹H NMR (500 MHZ, DMSO-d₆) δ 11.52 (d, J=12.6 Hz, 1H), 11.13 (d, J=28.8 Hz, 1H), 10.69 (s, 1H), 10.35 (s, 1H), 10.07 (s, 1H), 9.75 (s, 1H), 8.73 (s, 1H), 8.04 (d, J=8.8 Hz, 1H), 7.87 (d, J=8.7 Hz, 2H), 7.84-7.79 (m, 3H), 7.76 (s, 1H), 7.64 (s, 1H), 7.59 (d, J=8.8 Hz, 1H), 7.45 (d, J=10.6 Hz, 2H), 7.35 (d, J=8.3 Hz, 2H), 7.26 (s, 1H), 6.89-6.77 (m, 2H), 4.90-4.82 (m, 1H), 3.91 (s, 3H), 3.74 (s, 3H), 3.27-3.18 (m, 2H), 2.11 (s, 3H).

¹⁹F NMR (471 MHZ, DMSO-d₆) δ −112.8.

(¹H,¹³C)-HSQC_ed NMR (176 MHZ, DMSO-d₆) δ 110.6, 128.7, 119.5, 126.3, 119.5, 113.9, 119.0, 133.3, 126.2, 102.6, 131.8, 134.4, 129.6, 115.9, 115.6, 54.8, 60.6, 61.1, 27.9, 15.0, 22.5, 29.3.

HRMS (ESI-ORBITRAP): m/z [M+H]⁺ ber. für C₄₅H₃₉F₂N₈O₁₂: 921.2650, gef. 921.2683, R_{T}=8.09 min.

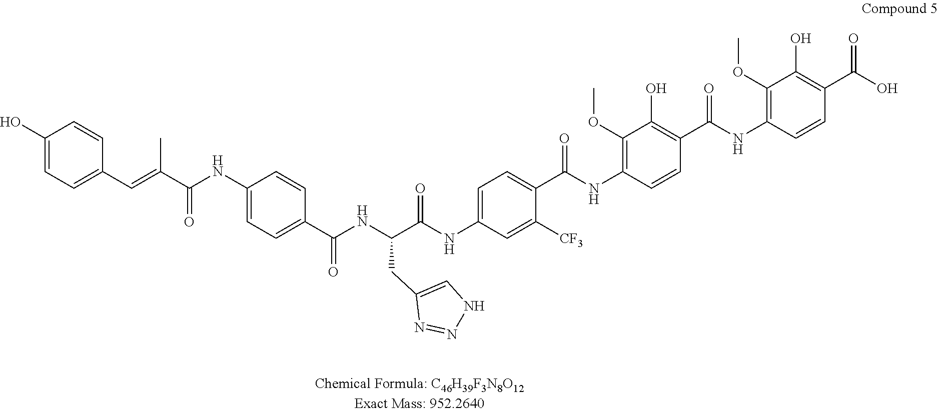 [SMILES: COc1c(NC(=O)c2ccc(NC(=O)c3ccc(NC(=O)[C@H](Cc4c[nH]nn4)NC(=O)c4ccc(NC(=O)/C(C)=C/c5ccc(O)cc5)cc4)cc3C(F)(F)F)c(OC)c2O)ccc(C(=O)O)c1O]

¹H NMR (500 MHZ, DMSO-d₆) δ 11.49 (d, J=12.5 Hz, 1H), 11.17 (s, 1H), 10.64 (s, 1H), 10.15 (s, 1H), 10.07 (s, 1H), 9.75 (s, 1H), 8.71 (d, J=7.4 Hz, 1H), 8.16 (d, J=1.8 Hz, 1H), 8.05 (d, J=8.8 Hz, 1H), 7.93 (d, J=8.5 Hz, 1H), 7.87 (d, J=8.6 Hz, 2H), 7.82 (d, J=8.6 Hz, 3H), 7.68 (d, J=8.3 Hz, 2H), 7.59 (d, J=8.9 Hz, 1H), 7.35 (d, J=8.3 Hz, 2H), 7.26 (s, 1H), 6.84 (d, J=8.1 Hz, 2H), 4.89 (s, 1H), 3.91 (s, 3H), 3.74 (s, 3H), 3.26 (d, J=8.9 Hz, 2H), 2.11 (s, 3H).

¹⁹F NMR (659 MHZ, DMSO-d₆) δ −58.10.

(¹H, ¹³C)-HSQC_ed NMR (176 MHz, DMSO-d₆) δ 116.9, 110.6, 122.6, 119.5, 128.7, 128.7, 119.5, 126.2, 130.2, 114.4, 126.2, 131.8, 134.4, 115.8, 54.7, 60.7, 61.1, 27.8, 15.0, 29.4.

HRMS (ESI-ORBITRAP): m/z [M+H]⁺ calcd. C₄₆H₄₀F₃N₈O₁₂: 953.2712, gef. 953.2717, R_{T}=8.35 min.

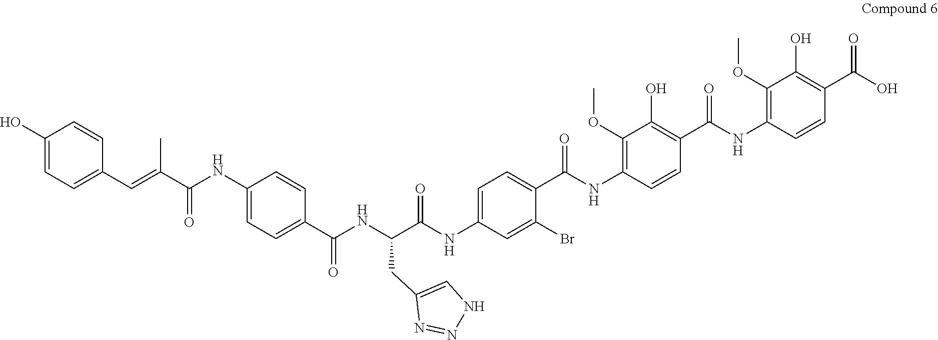 [SMILES: COc1c(NC(=O)c2ccc(NC(=O)c3ccc(NC(=O)[C@H](Cc4c[nH]nn4)NC(=O)c4ccc(NC(=O)/C(C)=C/c5ccc(O)cc5)cc4)cc3Br)c(OC)c2O)ccc(C(=O)O)c1O]

¹H NMR (500 MHZ, DMSO-d₆) δ 11.58 (s, 1H), 11.52 (s, 1H), 11.17 (s, 1H), 10.49 (s, 1H), 10.07 (s, 1H), 10.00 (s, 1H), 9.75 (s, 1H), 8.69 (d, J=7.4 Hz, 1H), 8.09-8.02 (m, 2H), 7.87 (d, J=8.6 Hz, 2H), 7.82 (d, J=8.4 Hz, 3H), 7.75 (s, 1H), 7.68 (s, 1H), 7.65-7.62 (m, 1H), 7.57 (dd, J=19.5, 8.6 Hz, 2H), 7.35 (d, J=8.3 Hz, 2H), 7.26 (s, 1H), 6.84 (d, J=8.3 Hz, 2H), 4.87 (d, J=7.6 Hz, 1H), 3.91 (s, 3H), 3.77 (s, 3H), 3.30-3.22 (m, 2H), 2.11 (s, 3H).

HRMS (ESI-ORBITRAP): m/z [M+H]⁺ ber. für C₄₅H₄₀BrN₈O₁₂: 963.1944, gef. 963.1949, R_{T}=7.93 min.

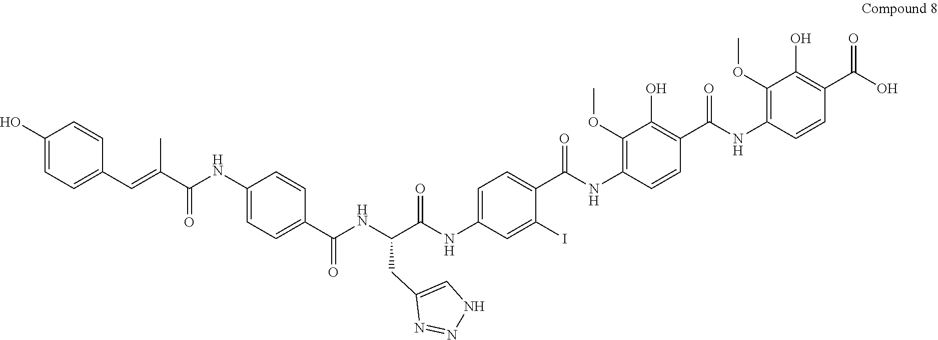 [SMILES: COc1c(NC(=O)c2ccc(NC(=O)c3ccc(NC(=O)[C@H](Cc4c[nH]nn4)NC(=O)c4ccc(NC(=O)/C(C)=C/c5ccc(O)cc5)cc4)cc3I)c(OC)c2O)ccc(C(=O)O)c1O]

¹H NMR (700 MHZ, DMSO-d₆) δ 11.58 (s, 1H), 11.52 (s, 1H), 11.19 (s, 1H), 10.42 (s, 1H), 10.08 (s, 1H), 9.96 (s, 1H), 9.76 (s, 1H), 8.68 (d, J=7.6 Hz, 1H), 8.28 (d, J=2.0 Hz, 1H), 8.06 (d, J=8.9 Hz, 1H), 7.88-7.84 (m, 2H), 7.81 (dt, J=9.2, 2.0 Hz, 3H), 7.76-7.71 (m, 1H), 7.67 (dd, J=8.4, 2.0 Hz, 1H), 7.59 (d, J=8.8 Hz, 1H), 7.46 (d, J=8.3 Hz, 1H), 7.35 (d, J=8.7 Hz, 2H), 7.26 (d, J=1.6 Hz, 1H), 6.84 (d, J=8.6 Hz, 2H), 4.86 (q, J=7.5 Hz, 1H), 3.91 (s, 3H), 3.78 (s, 3H), 3.29 (dd, J=14.8, 5.8 Hz, 1H), 3.22 (dd, J=14.7, 9.0 Hz, 1H), 2.11 (d, J=1.4 Hz, 3H).

HRMS (ESI-ORBITRAP): m/z [M+H]⁺ ber. für C₄₅H₄₀IN₈O₁₂: 1011.1805, gef. 1011.1799, R_{T}=8.39 min.

### Test for Biological Activity

### Strains:

E. coli BW25113, S. typhimurium TA100; Bacillus subtilis DSM10; and M. phlei DSM 750 and human serum

### Biological Testing:

The tests were performed using the micro dilution method.

### Microdilution Assay:

The determination of MIC values was performed according to the ninth edition of the Approved Standard M07-A9 (CLSI. Methods for Dilution Antimicrobial Susceptibility Tests for Bacteria That Grow Aerobically; Approved Standard-Ninth Edition. CLSI document M07-A9. Wayne, PA: Clinical and Laboratory Standards Institute; 2012.)

The test was carried out for different bacterial strains (E. coli DSM 1116, E. coli BW25113 [gram negative], B. subtilis DSM 10 [gram positive], M. luteus DSM 1790 [gram positive], S. typhimurium TA100 [gram negative]) and M. phlei DSM 750. 20 UL of cryo stock of each strain were inoculated in 20 mL of LB media (Lysogeny broth: 10 g/L peptone, 5 g/L yeast extract, 5 g/L NaCl) followed by incubation over night at 37° C., 200 rpm. The test inoculum was adjusted by the 0.5 McFarland Standard (OD625 from 0.08 to 0.1). Within 15 min of preparation, the adjusted inoculum suspension was diluted in MHBII media (BBL TM Mueller-Hinton Broth II, Becton, Dickinson and Company, New Jersey/USA) so that each well contained approximately 5×105 CFU/mL in a final volume of 100 μL. 95 μL of the inoculum were applied per well and 5 μL of the (diluted) antibiotic substance were added.

Previously the dry antibiotic compounds were dissolved in DMSO (100%) with a concentration of 2560 μg/mL and the resulting stock solutions were further diluted in DMSO (100%). 5 L of each antibiotic dilution were applied to the microdilution tray to reach final concentrations of 64 μg/mL to 0.008 μg/ml. One row of each well plate was left as a growth control without antibiotic substances and another row of the microdilution tray was used as sterility control (only MHB II-media). The antimicrobial effect of the solvent (DMSO) was tested by adding 5 μL DMSO to several wells without antibiotics. Purity check and cell titer control were performed according to International Standard M07-A9 on Mueller-Hinton II Agar (Mueller Hinton II Broth, 15 g/L agar-agar). Both microdilution trays and agar plates were incubated at 37° C. for 20 h and subsequently analyzed visually.

The results are summarized in table 1. The potency of an antibiotic is determined by the minimum inhibitory concentration (MIC). Contrary to intuition, a particularly low value is equated with a high potency.

The following compound (0) is used as reference and lead structure and is of the following structure:

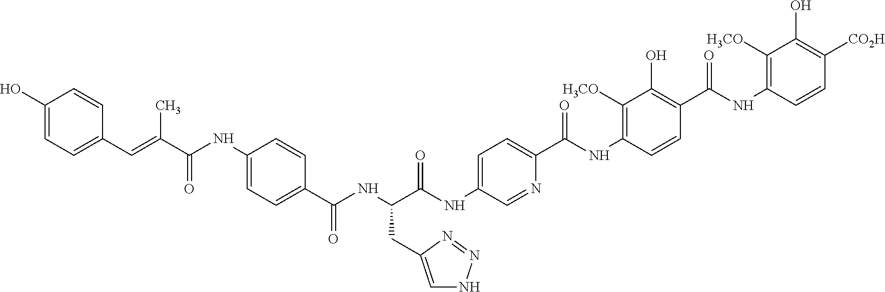 [SMILES: COc1c(NC(=O)c2ccc(NC(=O)c3ccc(NC(=O)[C@H](Cc4c[nH]nn4)NC(=O)c4ccc(NC(=O)/C(C)=C/c5ccc(O)cc5)cc4)cn3)c(OC)c2O)ccc(C(=O)O)c1O]

TABLE 1
Antibacterial activity of compounds according
to the solution against selected strains

| Compound | | 0 | 1 | 2 | 3 | 5 | 6 | 8 |
|---|---|---|---|---|---|---|---|---|
| Gram- | E. Coli BW25113 | 0.015 | 0.016 | 0.015 | 0.015 | 0.125 | 0.016 | 0.063 |
| negativ | S. Typhimirium | 0.015 | 0.015 | 0.015 | 0.015 | 0.031 | 0.016 | 0.031 |
| | 50% human serum | 0.5 | 1 | 0.5 | 0.5 | 9 | 1 | 9 |
| | E. Coli. | | | | | | | |
| Gram- | B. Subtilis | 0.031 | 0.125 | 0.125 | 0.063 | 4.0 | 0.125 | 0.5 |
| positiv | M. Luteus | 0.031 | 0.031 | 0.25 | 10.25 | 4.0 | 0.5 | 4 |
| | M. phlei | 0.125 | 1 | 1 | 0.5 | 8.0 | 1 | 4 |

[≥8.0 = 9; ≤0.016 = 0.015]

Among the D-variations in Table 1, compounds 2 (difluoro) and 3 (chloro) should be highlighted. Due to their electronegative substituents and the H-bonds formed as a result, they are about as active as the lead structure (0). In contrast, however, they have a higher steric hindrance in the space of the D-E amide bond, this could prevent the attack of the serine endopeptidase, especially from the larger CI ato

1. A compound having the general formula (1) 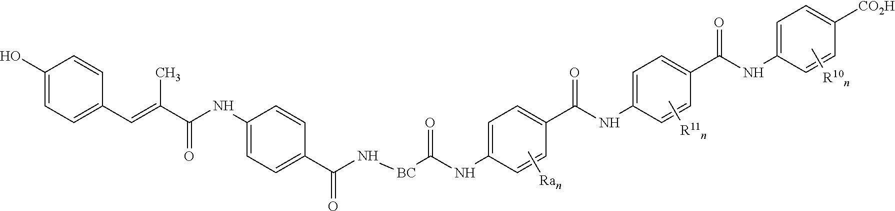
a) with BC being 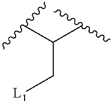
with L₁ being a substituted or unsubstituted C₅-C₆ aromatic heterocycle,
b) with n of R¹⁰ₙ and n of R¹¹ₙ being independently from each other 0, 1, 2, 3 or 4, in particular n of R¹⁰ₙ and n of R¹¹, being 0, 1, 2 or 3, more particular n of R¹⁰ₙ and n of R¹¹ₙ being 1 and 2;
with each R¹⁰ and R¹¹ being selected independently from any other R¹⁰ and R¹¹ from —OH, —F, —Cl, —Br, —I, —CCH, —CN, —OC₁-C₆ alkyl, in particular from —OH, —F, —OCH₃, —OC₂H₅, —OiC₃H₇, —OnC₃H₇, —OCF₃; and
c) with n of R^{a}ₙ being 1 or 2, with Rₐ being a substituent selected from halogen, in particular —Cl, —F, —Br, —I, —NO₂, —NH₂, —CO₂H, —CN, —CF₃.

2. The compound according to claim 1, wherein Rₐ being an electron withdrawing moiety selected from —Cl, —F, —Br, —I, —CF₃.

3. The compound according to claim 1, wherein L₁ is a five membered aromatic N-heterocycle, in particular a substituted or unsubstituted imidazole or triazole, most preferably a triazole.

4. The compound according to claim 1, wherein n of R¹⁰ₙ and n of R¹¹ₙ being 0, 1, 2, 3 or 4, in particular n of R¹⁰ₙ and n of R¹¹ₙ being 0, 1, 2 or 3, and with each R¹⁰ and with each R¹¹ independently from any other R¹⁰ being selected from —OH, —OCH₃, —OC₂H₅ or —OiPr, particularly with one R¹⁰ or R¹¹ being —OH and the other R¹⁰ or R¹¹ being —OCH₃, —OC₂H₅ or —OiPr respectively.

5. The compound according to claim 1, having the general formulae (2) 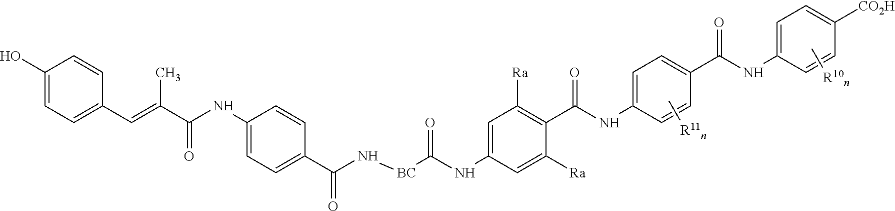
with R^{a} being a substituent selected from halogen, in particular —Cl, —F, —Br, —I, —NO₂, —NH₂, —CO₂H, —CN, —CF₃,
with BC, R¹⁰ₙ and R¹¹ₙ as in claim 1.

6. The compound according to claim 1, having the general formulae (3) 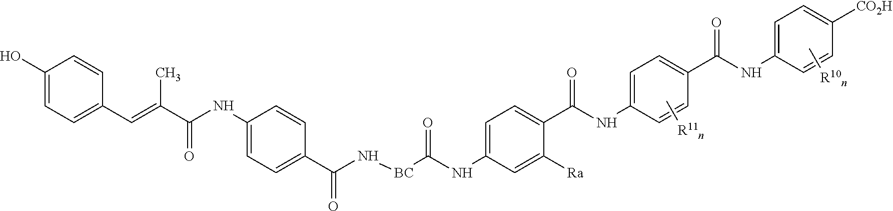
with R^{a} being a substituent selected from halogen, in particular —Cl, —F, —Br, —I, —NO₂, —NH₂, —CO₂H, —CN, —CF₃,
with BC, R¹⁰ₙ and R¹¹ₙ as in claim 1.

7. The compound according to claim 1, having the general formulae (4) 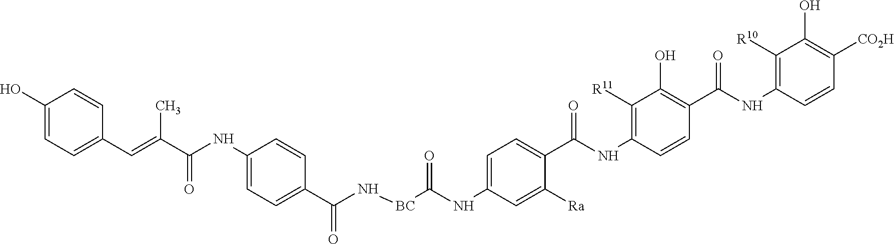
with R^{a} being a substituent selected from halogen, in particular —Cl, —F, —Br, —I, —NO₂, —NH₂, —CO₂H, —CN, —CF₃,
with BC, R¹⁰ and R¹¹ₙ as in claim 1.

8. The compound according to claim 1, having the general formulae (5) 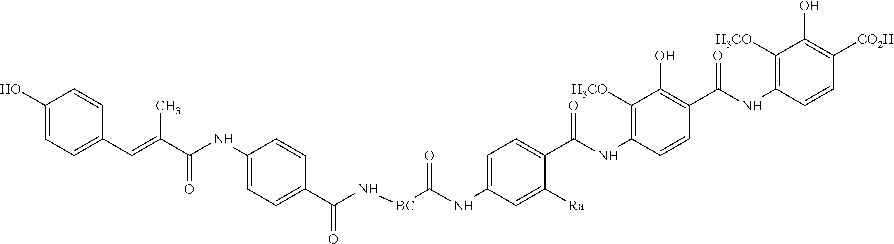
with R^{a} being a substituent selected from halogen, in particular —Cl, —F, —Br, —I, —NO₂, —NH₂, —CO₂H, —CN, —CF₃,
with BC as in claim 1.

9. A method of treatment of diseases of a patient in need thereof by administering a compound according to claim 1 to said patient.

10. The method according to claim 9, wherein the bacterial infection is an infection by one of the genus Acinetobacter, Bordatella, Borellia, Brucella, Camphylobacter, Chlamydia, Chlamydophila, Enterobacter, Escherichia, Francisella, Haemophilus, Helicobacter, Klebsiella, Legionella, Leptospira, Morganella Moraxella, Neisseria, Proteus, Pseudomonas, Rickettsia, Shigella, Salmonella, Stenotrophomonas, Treponema or Yersinia, Bacillus, Clostridium, Corynebacterium, Enterococcus, Listeria, Micrococcus, Staphylococcus or Streptococcus Mycobacterium, Mycoplasmataceae, in particular Escherichia, Bacillus, Salmonella, Micrococcus, Mycobacterium.

11. The method according to claim 9, wherein said treatment is a treatment of bacterial infections by gram-negative or gram-positive bacterial strains.
